(19) United States
(12) Patent Application Publication
(10) Pub. No.: US 2025/0161292 A1
(43) Pub. Date: May 22, 2025

(54) SOLID FORMS OF ((S)-5-((1-(6-CHLORO-2-OXO-1,2-DIHYDROQUINOLIN-3-YL)ETHYL)AMINO)-1-METHYL-6-OXO-1,6-DIHYDROPYRIDINE-2-CARBONITRILE

(71) Applicant: FORMA Therapeutics, Inc., Watertown, MA (US)

(72) Inventors: George P. Luke, Clinton, CT (US); Pratik Sheth, Watertown, MA (US)

(21) Appl. No.: 18/752,726

(22) Filed: Jun. 24, 2024

Related U.S. Application Data
(63) Continuation of application No. 18/215,727, filed on Jun. 28, 2023, now Pat. No. 12,053,463.
Application No. 18/215,727 is a continuation of application No. 17/183,606, filed on Feb. 24, 2021, now Pat. No. 11,723,905.
Application No. 17/183,606 is a continuation of application No. 16/693,642, filed on Nov. 25, 2019, now Pat. No. 10,959,994.
Application No. 16/693,642 is a continuation of application No. 16/414,716, filed on May 16, 2019, now Pat. No. 10,532,047.
(60) Provisional application No. 62/692,591, filed on Jun. 29, 2018.
(60) Provisional application No. 62/672,462, filed on May 16, 2018.
(60) Provisional application No. 62/672,461, filed on May 16, 2018.

(51) Int. Cl. A61K 31/4709; A61K 9/00; A61K 47/12; A61K 47/38

(52) U.S. Cl. CPC A61K 31/4709; A61K 9/0053; A61K 47/12; A61K 47/38

(57) ABSTRACT

The present disclosure reports solid forms of ((S)-5-((1-(6-chloro-2-oxo-1,2-dihydroquinolin-3-yl)ethyl)amino)-1-methyl-6-oxo-1,6-dihydropyridine-2-carbonitrile.

### CROSS REFERENCE TO RELATED APPLICATIONS

This application claims the benefit of and priority to U.S. provisional patent application No. 62/672,461, filed on May 16, 2018, U.S. provisional patent application No. 62/672,462, filed on May 16, 2018, and U.S. provisional patent application No. 62/692,591, filed on Jun. 29, 2018, the entire contents of which are incorporated herein by reference.

### TECHNICAL FIELD

The present disclosure relates to pharmaceutical compositions, including a solid form of a certain compound useful for inhibiting mutant isocitrate dehydrogenase 1 (mIDH1).

### BACKGROUND

The solid form of a compound may be important when the compound is used for pharmaceutical purposes. For example, compared with an amorphous solid, the solid physical properties of a crystalline compound may change from one solid form to another, which may affect its suitability for pharmaceutical use. In addition, different solid forms of a crystalline compound can incorporate different types and/or different amounts of impurities. The solid form of a compound can also affect chemical stability upon exposure to heat and/or water over a period of time.

The compound ((S)-5-((1-(6-chloro-2-oxo-1,2-dihydroquinolin-3-yl)ethyl)amino)-1-methyl-6-oxo-1,6-dihydropyridine-2-carbonitrile (“Compound 1”)

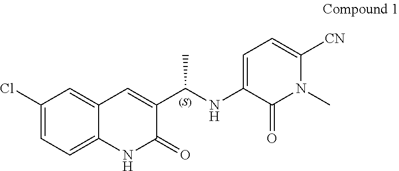

is an selective inhibitor of R132X mIDH-1. The free base of Compound 1 can be formulated into a pharmaceutical composition for treating patients diagnosed with a mIDH-1 form of cancer. The pharmaceutical composition can be provided in a unit dosage form (e.g., a capsule or unit dosage form) for oral use.

A preparation of a lyophilized solid form of Compound 1 is described in the publication WO2016/044789. However, therapeutic compounds often exist in a variety of solid forms having different properties. There remains a need for identifying solid forms of Compound 1 useful for various therapeutic applications.

### SUMMARY

Crystalline ((S)-5-((1-(6-chloro-2-oxo-1,2-dihydroquinolin-3-yl)ethyl)amino)-1-methyl-6-oxo-1,6-dihydropyridine-2-carbonitrile (Compound 1), and methods of making compositions comprising crystalline Compound 1, are disclosed herein. In some embodiments, a novel solid form of Compound 1 disclosed herein includes Compound 1 in a solid form designated as Type A, as well as compositions comprising a solid form of Compound 1. Novel compositions also include Compound 1 as a solid form designated as Type A, and/or crystalline and amorphous solid forms of Compound 1. The various solid forms of Compound 1 can be identified by certain characteristic properties.

A preferred solid form of ((S)-5-((1-(6-chloro-2-oxo-1,2-dihydroquinolin-3-yl)ethyl)amino)-1-methyl-6-oxo-1,6-dihydropyridine-2-carbonitrile (Compound 1) can be characterized by a reflection X-ray powder diffraction (XRPD) pattern comprising characteristic peaks at 6.3, 12.8, 13.8, 23.6, and 27.8 degrees±0.2° 2θ.

### BRIEF DESCRIPTION OF THE DRAWINGS

FIG. 1 depicts a dynamic vapor sorption (DVS) isotherm plot of Compound 1 Type A solid form.

FIG. 2 depicts a differential scanning calorimetry (DSC) thermogram for Compound 1 Type A solid form.

FIG. 3 depicts a thermogravimetric analysis (TGA) curve for Compound 1 Type A solid form.

FIG. 4 depicts X-ray powder diffraction (XRPD) pattern of Compound 1 Type A solid form.

### DETAILED DESCRIPTION

The bioactive chemical ((S)-5-((1-(6-chloro-2-oxo-1,2-dihydroquinolin-3-yl)ethyl)amino)-1-methyl-6-oxo-1,6-dihydropyridine-2-carbonitrile can be prepared as a solid form. The compound ((S)-5-((1-(6-chloro-2-oxo-1,2-dihydroquinolin-3-yl)ethyl)amino)-1-methyl-6-oxo-1,6-dihydropyridine-2-carbonitrile (“Compound 1”) is shown below:

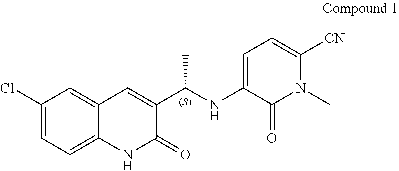

Compound 1 can also be referred to as olutasidenib, CAS No.: 1887014-12-1, (S)-5-((1-(6-chloro-2-oxo-1,2-dihydroquinolin-3-yl)ethyl)amino)-1-methyl-6-oxo-1,6-dihydropyridine-2-carbonitrile, or 5-{[(1S)-1-(6-chloro-2-oxo-1,2-dihydroquinolin-3-yl)ethyl]amino}-1-methyl-6-oxo-1,6-dihydropyridine-2-carbonitrile.

Compound 1 can occur in an amorphous solid form or in a crystalline solid form or in mixtures of solid forms. Crystalline solid forms of Compound 1 can exist in one or more unique solid forms, which can additionally comprise one or more equivalents of water or solvent (i.e., hydrates or solvates, respectively).

As disclosed herein, crystalline form(s) of Compound 1 have distinct characteristic XRPD peaks (see Example 2) that are not characterized in previous disclosures of Compound 1. Accordingly, provided herein are crystalline Compound 1 solid forms, pharmaceutical compositions thereof, and methods of preparing those crystalline Compound 1 solid forms and methods of use thereof.

A novel Compound 1 solid form can be obtained by a method reported in Example 2. For example, Compound 1 Type A can be prepared by a solution comprising Compound 1 and a solvent, and concentrating the solution, then diluting the solution with a solvent, stirring the solution for a period of time and cooling the solution to precipitate a crystalline solid of Compound 1 Type A. In some embodiments, the solution is stirred at room temperature. In some embodiments, the solution is cooled to a temperature of about 0° C. In some embodiments, the solvent comprises ethyl acetate.

As used herein, the term “precipitate” refers to the formation of a solid substance from a solution containing the same substance. A substance which precipitates from solution may be amorphous or crystalline. Precipitation may occur under a variety of conditions known to those of skill in the art, including the treatment of a solution of a solute (e.g., solute A in solvent B) with an antisolvent (i.e., a solvent that is miscible with solvent B, but does not dissolve solute A).

Solid forms of Compound 1 can be identified by various analytical techniques, such as X-ray powder diffraction (XRPD). Solid forms of Compound 1 disclosed herein include Compound 1 Type A solid form, as well as compositions comprising a solid form of Compound 1 comprising Type A solid form.

A novel Compound 1 Type A solid form is characterized by an X-ray Powder Diffraction (XRPD) pattern, having diffraction peaks at angles (2 theta±0.2) of 6.3, 12.8, 13.8, 23.6, and 27.8. In some embodiments, a novel Compound 1 Type A is characterized by an X-ray Powder Diffraction (XRPD) pattern, having diffraction peaks at angles (2 theta±0.2) of 6.3, 12.8, 13.8, 23.6, and 27.8, corresponding to d-spacing (angstroms±0.2) of 14.0, 6.9, 6.4, 3.8, and 3.2, respectively. In some embodiments, Compound 1 Type A can be identified by X-ray Powder Diffraction (XRPD) pattern, having characteristic diffraction peaks at angles (2 theta±0.2) of 5.7, 6.3, 8.5, 10.6, 12.8, 13.8, 17.3, 22.0, 22.8, 23.6, and 27.8. In some embodiments, Compound 1 Type A can be identified by X-ray Powder Diffraction (XRPD) pattern, having characteristic diffraction peaks at angles (2 theta±0.2) of 5.7, 6.3, 8.5, 10.6, 12.8, 13.8, 17.3, 22.0, 22.8, 23.6, and 27.8, corresponding to d-spacing (angstroms±0.2) of 15.4, 14.0, 8.4, 6.9, 6.4, 5.1, 4.0, 3.9, 3.8, and 3.2, respectively.

In some embodiments, Compound 1 Type A solid form is characterized by an X-ray powder diffraction having peaks at the same or substantially the same angles (2θ±0.2) and corresponding d-spacing (Å±0.2) of:

| | d-spacing |
|---|---|
| 2θ ± 0.2 | Å ± 0.2 |
| 5.7 | 15.4 |
| 6.3 | 14.0 |
| 8.5 | 10.4 |
| 10.6 | 8.4 |
| 11.4 | 7.8 |
| 12.8 | 6.9 |
| 13.8 | 6.4 |
| 14.2 | 6.2 |
| 15.2 | 5.8 |
| 15.6 | 5.7 |
| 17.3 | 5.1 |
| 17.9 | 5.0 |
| 18.2 | 4.9 |
| 18.9 | 4.7 |
| 19.6 | 4.5 |
| 20.6 | 4.3 |
| 21.5 | 4.1 |
| 22.0 | 4.0 |
| 22.8 | 3.9 |
| 23.6 | 3.8 |
| 24.5 | 3.6 |
| 24.8 | 3.6 |
| 25.3 | 3.5 |
| 25.6 | 3.5 |
| 26.0 | 3.4 |
| 26.3 | 3.4 |
| 27.0 | 3.3 |
| 27.8 | 3.2 |
| 28.9 | 3.1 |
| 30.0 | 3.0 |
| 31.2 | 3.0 |
| 32.1 | 2.8 |
| 33.6 | 2.7 |
| 34.1 | 2.6 |
| 36.3 | 2.5 |
| 37.0 | 2.4 |
| 38.1 | 2.4 |

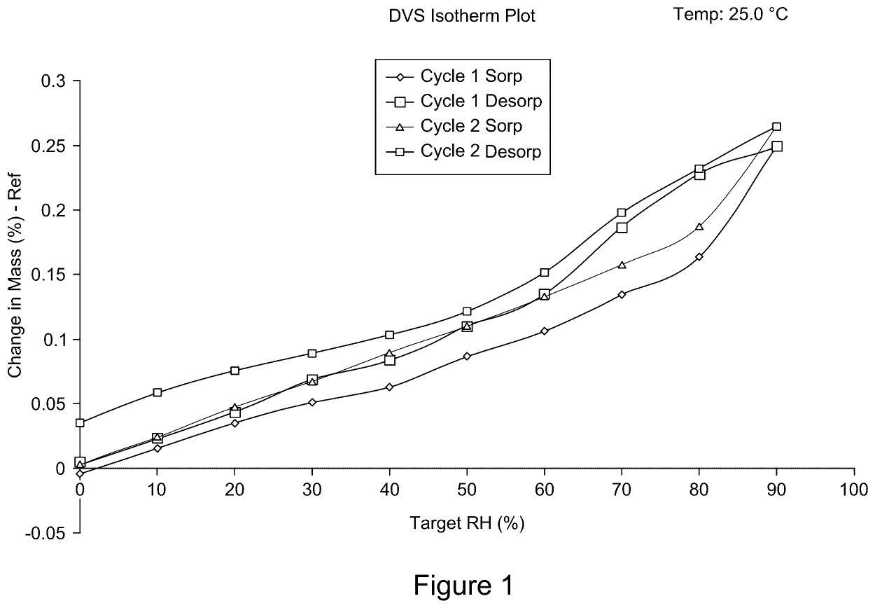

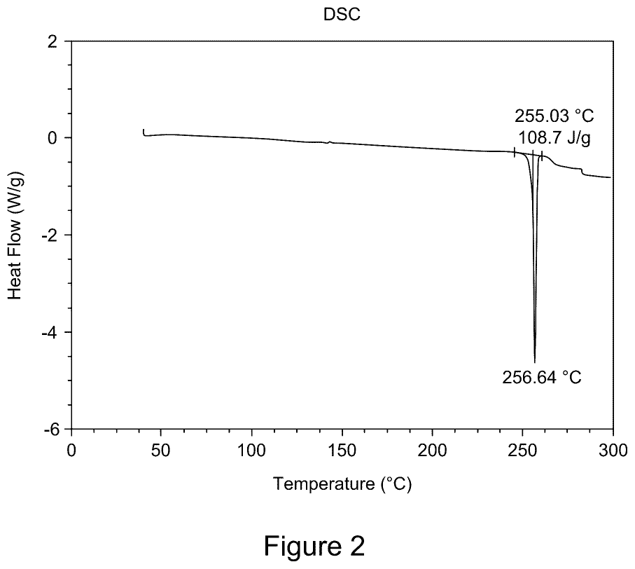

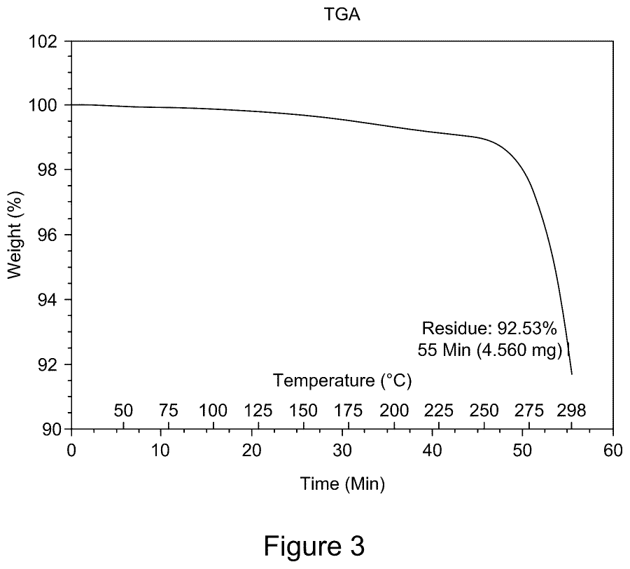

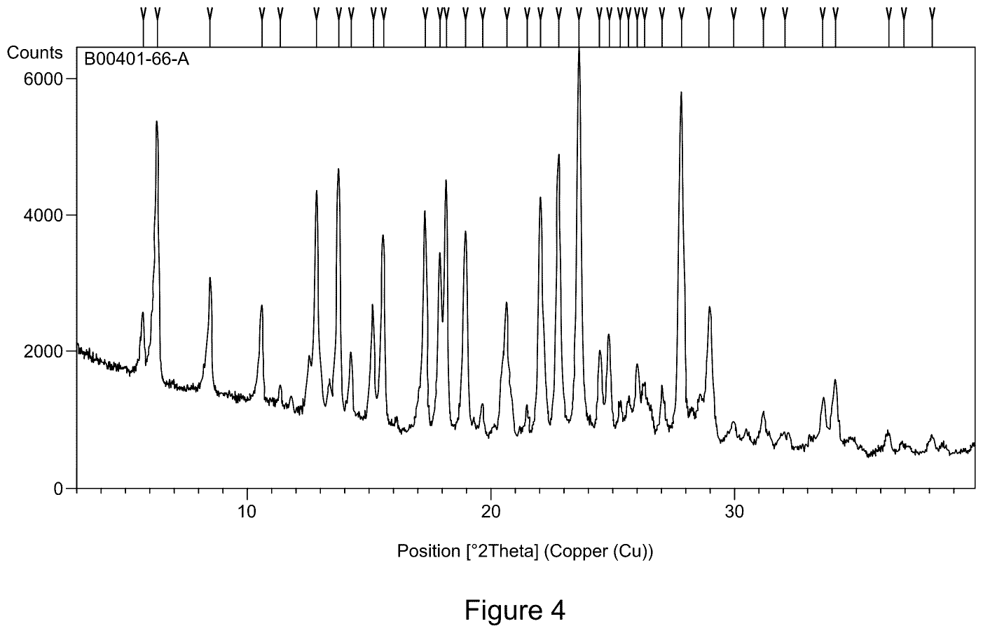

The presence of Compound 1 in the Type A solid form can be identified by one or more techniques, including DSC, TGA, DVS and XRPD. In some embodiments, Compound 1 Type A solid form is characterized by a differential scanning calorimetry (DSC) endotherm having a minima at about 256.64° C. The dynamic vapor sorption (DVS) isotherm plot of FIG. 1, the differential scanning calorimetry (DSC) thermogram of FIG. 2, the thermogravimetric analysis (TGA) plot in FIG. 3 and the X-ray Powder Diffraction (XRPD) pattern in FIG. 4 were each obtained by a composition comprising Compound 1, Type A solid form. The presence of Compound 1 Type A solid form can be identified by performing DSC, TGA, DVS and/or XRPD analysis of a composition and identifying sufficient similarity in comparison with the DVS isotherm plot in FIG. 1, the DSC thermogram in FIG. 2, the TGA plot in FIG. 3 and/or the XRPD pattern of FIG. 4.

In some embodiments, a pharmaceutical composition can comprise, and/or be obtained from, Compound 1 Type A solid form characterized by a dynamic vapor sorption (DVS) isotherm plot substantially similar to FIG. 1. In some embodiments, a pharmaceutical composition can comprise, and/or be obtained from, Compound 1 Type A solid form characterized by a differential scanning calorimetry (DSC) thermogram substantially similar to FIG. 2. In some embodiments, a pharmaceutical composition can comprise, and/or be obtained from, Compound 1 Type A solid form characterized by a thermogravimetric analysis (TGA) plot substantially similar to FIG. 3. In some embodiments, a pharmaceutical composition can comprise, and/or be obtained from, Compound 1 Type A solid form characterized by an X-ray Powder Diffraction (XRPD) pattern substantially similar to FIG. 4.

In some embodiments, the present disclosure provides a composition comprising amorphous and crystalline solid forms of Compound 1. In some embodiments, the composition comprises crystalline Compound 1 and amorphous Compound 1, wherein the amorphous Compound 1 is present in an amount selected from the following ranges: 90-99%, 80-90%, 70-80%, 60-70%, 50-60%, 40-50%, 30-40%, 20-30%, 10-20%, 1-10% and 0-1%.

In some embodiments, the present disclosure provides a pharmaceutical composition such as a drug product or drug substance comprising a solid form of Compound 1 disclosed herein. In some embodiments, the present disclosure provides a pharmaceutical composition comprising crystalline solid Type A of Compound 1. For example, a pharmaceutical composition can comprise, and/or be obtained from, the solid form of Compound 1 designated as Type A solid form of Compound 1 and is characterized by an X-ray Powder Diffraction (XRPD) pattern, having characteristic diffraction peaks at angles (2 theta±0.2) of 6.3, 12.8, 13.8, 23.6, and 27.8. In some embodiments, a pharmaceutical composition can comprise, and/or be obtained from, the solid form of Compound 1 designated as Type A of Compound 1 and is characterized by an XRPD pattern having characteristic diffraction peaks at angles (2 theta±0.2) of 5.7, 6.3, 8.5, 10.6, 12.8, 13.8, 17.3, 22.0, 22.8, 23.6, and 27.8. In some embodiments, a pharmaceutical composition can comprise, and/or be obtained from, the solid form of Compound 1 Type A and is characterized by an X-ray Powder Diffraction (XRPD) pattern, having diffraction peaks at angles (2 theta±0.2) of 6.3, 12.8, 13.8, 23.6, and 27.8, corresponding to d-spacing (angstroms±0.2) of 14.0, 6.9, 6.4, 3.8, and 3.2, respectively. In some embodiments, a pharmaceutical composition can comprise, and/or be obtained from, the solid form of Compound 1 Type A and can be identified by X-ray Powder Diffraction (XRPD) pattern, having characteristic diffraction peaks at angles (2 theta±0.2) of 5.7, 6.3, 8.5, 10.6, 12.8, 13.8, 17.3, 22.0, 22.8, 23.6, and 27.8, corresponding to d-spacing (angstroms±0.2) of 15.4, 14.0, 8.4, 6.9, 6.4, 5.1, 4.0, 3.9, 3.8, and 3.2, respectively.

In some embodiments, a pharmaceutical composition can comprise, and/or be obtained from, the solid form of Compound 1 designated as Type A of Compound 1 and is characterized by an XRPD pattern having peaks at substantially the same angles (2θ±0.2) and corresponding d-spacing (Å±0.2) of:

| | d-spacing |
|---|---|
| 2θ ± 0.2 | Å ± 0.2 |
| 5.7 | 15.4 |
| 6.3 | 14.0 |
| 8.5 | 10.4 |
| 10.6 | 8.4 |
| 11.4 | 7.8 |
| 12.8 | 6.9 |
| 13.8 | 6.4 |
| 14.2 | 6.2 |
| 15.2 | 5.8 |
| 15.6 | 5.7 |
| 17.3 | 5.1 |
| 17.9 | 5.0 |
| 18.2 | 4.9 |
| 18.9 | 4.7 |
| 19.6 | 4.5 |
| 20.6 | 4.3 |
| 21.5 | 4.1 |
| 22.0 | 4.0 |
| 22.8 | 3.9 |
| 23.6 | 3.8 |
| 24.5 | 3.6 |
| 24.8 | 3.6 |
| 25.3 | 3.5 |
| 25.6 | 3.5 |
| 26.0 | 3.4 |
| 26.3 | 3.4 |
| 27.0 | 3.3 |
| 27.8 | 3.2 |
| 28.9 | 3.1 |
| 30.0 | 3.0 |
| 31.2 | 3.0 |
| 32.1 | 2.8 |
| 33.6 | 2.7 |
| 34.1 | 2.6 |
| 36.3 | 2.5 |
| 37.0 | 2.4 |
| 38.1 | 2.4 |

Pharmaceutical compositions reported herein can be combined with a pharmaceutically acceptable carrier or excipient. In some embodiments, pharmaceutical compositions reported herein can be provided in a unit dosage form container (e.g., in a vial or bag or the like). In some embodiments, pharmaceutical compositions reported herein can be provided in an oral dosage form.

In some embodiments, the pharmaceutical composition comprises an active pharmaceutical ingredient (API) comprising, consisting essentially of, or consisting of Compound 1 prepared under applicable Good Manufacturing Practice (GMP). For example, the pharmaceutical composition can be a batch composition comprising Compound 1 (preferably including Solid Form Type A of Compound 1), wherein the batch composition adheres to Good Manufacturing Practices (e.g., ICH Harmonised Tripartite Guideline, Good Manufacturing Practice Guide for Active Pharmaceutical Ingredients Q7, Current Step 4 version dated 10 Nov. 2010). More preferably, the GMP batch composition can be a homogenous blended batch comprising Type A Solid Form of Compound 1. The FDA (Food and Drug Administration) provides applicable guidance on Good Manufacturing Practice (GMP) for the manufacturing of active pharmaceutical ingredients (APIs) under an appropriate system for managing quality. As used with respect to manufacture of API under GMP, “manufacturing” is defined to include all operations of receipt of materials, production, packaging, repackaging, labelling, relabelling, quality control, release, storage and distribution of APIs and the related controls. An “API Starting Material” is a raw material, intermediate, or an API that is used in the production of an API and that is incorporated as a significant structural fragment into the structure of the API. An API Starting Material can be an article of commerce, a material purchased from one or more suppliers under contract or commercial agreement, or produced in-house. API Starting Materials normally have defined chemical properties and structure.

In some embodiments, an oral dosage form of Compound 1 Type A can be a capsule. In some embodiments, an oral dosage form of Compound 1 Type A is a tablet. In some embodiments, an oral dosage form comprises a filler. In some embodiments, an oral dosage form comprises two fillers. In some embodiments, an oral dosage form comprises one or more fillers. In some embodiments, the filler is selected from the group consisting of Avicel PH101 (50 μm) and Avicel PH102 (100 μm). In some embodiments, an oral dosage form comprises one or more disintegrants. In some embodiments a disintegrant is Ac-Di-Sol. In some embodiments, the oral dosage form comprises one or more lubricants. In some embodiments, the lubricant is magnesium stearate. In some embodiments, an oral dosage form comprises one or more glidants, anti-adherents and/or anti-statics. In some embodiments, the glidant, anti-adherent and/or anti-static is colloidal silicon dioxide. In some embodiments,an oral dosage form is prepared via dry blending. In some embodiments, an oral dosage form is a tablet and is prepared via dry granulation.

In some embodiments, the present disclosure provides methods of inhibiting mutant isocitrate dehydrogenase 1 (mIDH1), comprising administering a solid form of Compound 1 to a subject. In some embodiments, the present disclosure provides methods of treating a disease, disorder, or condition responsive to inhibition of mutant isocitrate dehydrogenase 1 (mIDH1), comprising administering a solid form of Compound 1 (e.g., Compound 1 as a Type A solid form) to a subject in need thereof. In some embodiments, the disease, disorder, or condition is associated with mutant isocitrate dehydrogenase.

In some embodiments, the present disclosure provides methods of treating cancer comprising administering a solid form of Compound 1 (e.g., Compound 1 as a Type A solid form) to a subject in need thereof. In some embodiments, the present disclosure provides methods of reducing 2-hydroxyglutarate comprising administering a solid form of Compound 1 (e.g., Compound 1 as a Type A solid form) to a subject in need thereof. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

### EXAMPLES

### Instrumentation and Methods

Unless otherwise indicated, the following instrumentation and methods were used in the working examples described herein.

Unless otherwise noted, reagents and solvents were used as received from commercial suppliers. Proton nuclear magnetic resonance (NMR) spectra were obtained on either Bruker or Varian spectrometers at 300 MHz. Spectra are given in ppm (δ) and coupling constants, J, are reported in Hertz. Tetramethylsilane (TMS) was used as an internal standard. Mass spectra were collected using a Waters ZQ Single Quad Mass Spectrometer (ion trap electrospray ionization (ESI)). High performance liquid chromatograph (HPLC) analyses were obtained using a XBridge Phenyl or C18 column (5 μm, 50×4.6 mm, 150×4.6 mm or 250×4.6 mm) with UV detection (Waters 996 PDA) at 254 nm or 223 nm using a standard solvent gradient program (Methods 1-2).

### LCMS Method 1 (ESI, 4 Min Method):

### Instruments and Conditions:

### HPLC: Waters HT2790 Alliance MS: Waters ZQ Single Quad Mass Spectrometer

### UV: Waters 996 PDA

### Conditions:

Mobile phase A 95% water/5% methanol with 0.1% Formic Acid
Mobile phase B (B) 95% methanol/5% water with 0.1% Formic Acid

### Column XBridge Phenyl or C18, 5 μm 4.6×50 mm

Column temperature Ambient
LC gradient Linear 5-95% B in 2.5 min, hold 95% B to 3.5 min
LC Flow rate 3 mL/min
UV wavelength 220 nm and 254 nm
Ionization Mode Electrospray Ionization; positive/negative

### LCMS Method 2: (APCI, 20 Min)

### Instruments and Conditions:

HPLC-Agilent 1100 series.
Column: Agela Technologies Durashell C18, 3 μm, 4.6×50 mm,).

### Mobile Phase A: ACN+0.1% TFA.

### Mobile Phase B: Water+0.1% TFA.

| Gradient: Time (min) | % B |
|---|---|
| 00 | 95 |
| 15 | 05 |
| 18 | 05 |
| 20 | 95 |

Flow Rate: 1 mL/min.

### ColumnTemperature: Ambient.

### Detector: 254 nm.

### X-Ray Powder Diffraction (XRPD)

High resolution X-ray Powder Diffraction experiments were performed with a Panalytical X'Pert³ Powder X-ray diffractometer on a Si zero-background holder. The 2θ position was calibrated against Panalytical 640 Si powder standard. Details of the XRPD method are listed below:

Parameters for Reflection Mode

| X-Ray wavelength | Cu, kα, Kα1 (Å): 1.540598, Kα2 (Å): 1.544426 |
|---|---|
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0131 |
| Scan speed (°/s) | 0.033 |

Peaks are reported as diffraction angles at 2 theta, with d-spacing measured in angstroms.

### Thermal Analysis

Thermo gravimetric analysis (TGA) experiments were performed on TA Q500 TGA from TA Instruments. Samples were heated at 10° C./min from about 20° C. to about 300° C. using dry nitrogen to purge the system. The details of the method are provided below:

| Parameters | TGA |
|---|---|
| Pan Type | Platinum plate, open |
| Temperature | RT-250° C. |
| Ramp rate | 10° C./min |
| Purge gas | N₂ |

Differential scanning calorimetry (DSC) experiments were performed on a TA Q2000 DSC from TA Instruments. Samples were heated at 10° C./min from about 20° C. to about 300° C. using dry nitrogen to purge the system. The details of the method are provided below:

| Parameters | DSC |
|---|---|
| Pan Type | Aluminum pan, closed |
| Temperature | RT-250° C. |
| Ramp rate | 10° C./min |
| Purge gas | N₂ |

### Dynamic Vapor Sorption

Dynamic vapor sorption (DVS) was obtained using a Surface Measurement Systems (SMS) DVS Intrinsic. The details of the method are provided below:

| Parameters | Values | |
|---|---|---|
| Temperature | 25° | C. |
| Sample size | 10-20 | mg |
| Gas and flow rate | N₂, 200 mL/min | |
| dm/dt | 0.002%/min | |
| Min. dm/dt stability duration | 10 | min |
| Max. equilibrium time | 360 | min |

| | |
|---|---|
| RH range | 20% RH - 95% RH-0% RH-95% RH |
| RH step size | 10% (90% RH-0% RH-90% RH) |
| | 5% (95% RH-90% RH and 90% RH-95% RH) |

### Example 1—Synthesis of (S)-5-((1-(6-chloro-2-oxo-1,2-dihydroquinolin-3-yl)ethyl)amino)-1-methyl-6-oxo-1,6-dihydropyridine-2-carbonitrile (Compound 1)

Intermediate 1: (S)-3-(1-aminoethyl)-6-chloroquinolin-2(1H)-one hydrochloride

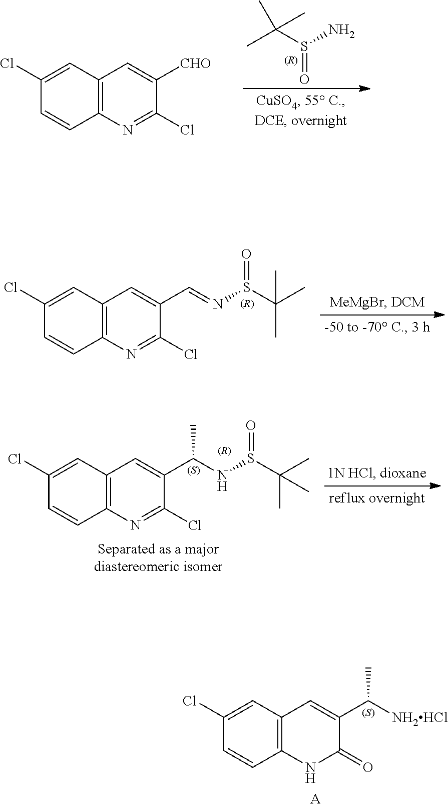

Step-1: (R,E)-N-((2,6-dichloroquinolin-3-yl)methylene)-2-methylpropane-2-sulfinamide

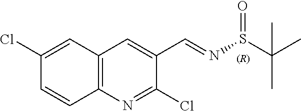

To a mixture of 2,6-dichloroquinoline-3-carbaldehyde (15.0 g, 66.37 mmol) and (R)-2-methylpropane-2-sulfinamide (8.85 g, 73.14 mmol) in 1,2-dichloroethane (150 mL) was added CuSO₄ (16.0 g, 100.25 mmol). The resulting mixture was heated to 55° C. and stirred at 55° C. overnight. After TLC and MS showed complete disappearance of starting materials, the mixture was cooled to room temperature and filtered through a pad of Celite®. The pad of Celite® was then rinsed with CH₂Cl₂. The filtrate was evaporated to dryness in vacuo and purified by SiO₂ column chromatography (0 to 25% hexanes/EtOAc) to afford the title compound, (R,E)-N-((2,6-dichloroquinolin-3-yl)methylene)-2-methylpropane-2-sulfinamide, as a yellow solid (17.7 g, 81% yield).

Step-2: (R)-N-((S)-1-(2,6-dichloroquinolin-3-yl)ethyl)-2-methylpropane-2-sulfinamide.

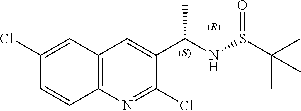

To a solution of (R,E)-N-((2,6-dichloroquinolin-3-yl)methylene)-2-methylpropane-2-sulfinamide (8.85 g, 26.88 mmol) in anhydrous CH₂Cl₂ (200 mL) at −60° C. was added dropwise MeMgBr (3M solution in diethyl ether, 13.5 mL, 40.54 mmol). The resulting reaction mixture was stirred at about −60 to −50° C. for 3 hours and then stirred at −20° C. overnight under an atmosphere of N₂. After TLC and MS showed complete disappearance of starting materials, saturated NH₄Cl (163 mL) was added at −20° C. and the resulting mixture was stirred for 10 minutes. The aqueous phase was extracted with CH₂Cl₂ (100 mL×3), dried over anhydrous Na₂SO₄, filtered, and evaporated. The residue was purified by column chromatography on an ISCO® chromatography system (SiO₂: Gold column; gradient; hexanes to 100% EtOAc) to provide the title compound, (R)-N-((S)-1-(2,6-dichloroquinolin-3-yl)ethyl)-2-methylpropane-2-sulfinamide, as a yellow solid (5.8 g, 63% yield).

Step-3: (S)-3-(1-aminoethyl)-6-chloroquinolin-2(1H)-one hydrochloride (A).

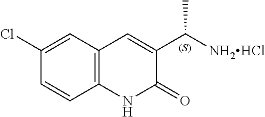

A mixture of (R)-N-((S)-1-(2,6-dichloroquinolin-3-yl)ethyl)-2-methylpropane-2-sulfinamide (6.6 g, 19.13 mmol) in 1,4-dioxane (41 mL) and 1N HCl (41 mL) was heated at reflux overnight. The solvents were evaporated in vacuo and the resulting residue was dissolved in hot water and lyophilized. The crude product was triturated with diethyl ether to afford the title compound A as a yellow solid (9.0 g, ee: 98.4%). ¹H NMR (300 MHz, DMSO-d₆): δ ppm 12.4 (br s, 1 H), 8.32 (br s, 2 H), 8.07 (s, 1 H), 7.85 (d, J=2.2 Hz, 1 H), 7.63 (dd, J1=8.8 Hz, J2=2.5 Hz, 1 H), 7.40 (d, J=8.8 Hz, 1 H), 4.40-4.45 (m, 1 H), 1.53 (d, J=8.5 Hz, 3 H). LCMS (Method 2): Rt 3.42 min, m/z 223.1 [M+H]⁺.

Intermediate 2: 5-fluoro-1-methyl-6-oxo-1,6-dihydropyridine-2-carbonitrile

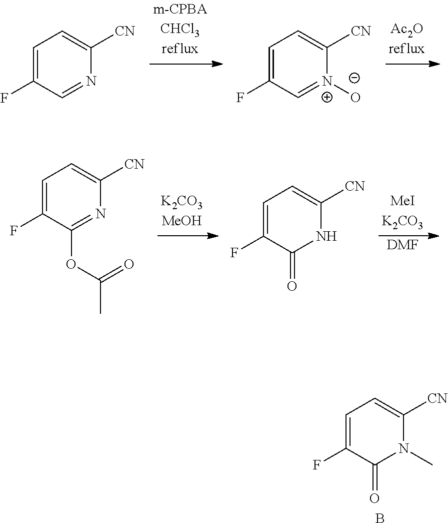

Step-1: 2-cyano-5-fluoropyridine 1-oxide

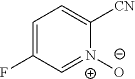

A solution of 5-fluoropicolinonitrile (7.27 g, 59.5 mmol) in CHCl₃ (60 mL) was added dropwise by addition funnel to a solution of m-CPBA (<77%, 22.00 g, 98 mmol) in CHCl3 160 mL). The solution was stirred at reflux for 4 days, at which time LCMS showed ˜85% conversion. The sample was allowed to cool, then sodium sulfite (12.4 g, 98 mmol) was added and the sample was stirred at room temperature for three hours, during which time the solution became thick with a white precipitate. The sample was diluted with DCM (300 mL) and filtered on a Buchner funnel, and the filter cake was washed with DCM (˜400 mL). A white material precipitated in the filtrate. The filtrate mixture was washed with saturated aqueous NaHCO₃ (400 mL), during which the solids went into solution. The organic layer was washed with water (300 mL), then dried (MgSO₄) and filtered. Silica gel was added and the mixture was evaporated under reduced pressure. The material was chromatographed by Biotage MPLC (340 g silica gel column) with 0 to 100% EtOAc in hexanes, with isocratic elution when peaks came off to provide 2-cyano-5-fluoropyridine 1-oxide (4.28 g, 31.0 mmol, 52% yield) as a white solid. ¹H NMR (300 MHz, DMSO-d₆): δ ppm 8.85-8.93 (m, 1 H), 8.23 (dd, J=9.09, 6.74 Hz, 1 H), 7.53-7.64 (m, 1 H). LCMS (Method 1): Rt 0.57 min., m/z 138.9 [M+H]⁺.

Step 2: 6-cyano-3-fluoropyridin-2-yl acetate

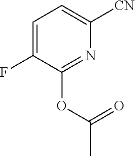

A solution of 2-cyano-5-fluoropyridine 1-oxide (4.28 g, 31.0 mmol) in acetic anhydride (40 ml, 424 mmol) was heated at reflux (150° C. bath) for three days, during which the clear solution turned dark. The sample was concentrated under reduced pressure. The residue was dissolved in MeOH (30 mL) and stirred for 1 hour. Silica gel was added and the solvent was evaporated under reduced pressure. The material was chromatographed by Biotage MPLC (100 g silica gel column) with 0 to 23% EtOAc in hexanes to provide 6-cyano-3-fluoropyridin-2-yl acetate (3.32 g, 18.43 mmol, 60% yield) as a clear liquid that solidified on cooling. ¹H NMR (300 MHz, CHLOROFORM-d): δ ppm 7.65-7.75 (m, 2 H), 2.42 (s, 3 H). LCMS (Method 1): Rt 1.54 min., m/z 138.8 (loss of acetate).

Step 3: 5-fluoro-6-oxo-1,6-dihydropyridine-2-carbonitrile

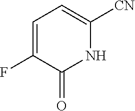

A solution of 6-cyano-3-fluoropyridin-2-yl acetate (3.32 g, 18.43 mmol) in MeOH (40 ml) was treated with potassium carbonate (5.10 g, 36.9 mmol) and stirred at room temperature for four hours. LCMS at 2 hours showed the reaction had gone to completion. The solvent was evaporated under reduced pressure. The residue was dissolved in water (100 mL) and acidified to pH≤1 with 1M HCl. The solution was extracted with EtOAc (3×100 mL). The combined organic extracts were dried (Na₂SO₄), filtered, and evaporated under reduced pressure to provide 5-fluoro-6-oxo-1,6-dihydropyridine-2-carbonitrile (2.34 g, 16.94 mmol, 92% yield) as a white solid. ¹H NMR (300 MHz, DMSO-d₆): δ ppm 12.92 (br s, 1 H), 7.73 (br s, 1 H), 7.43 (br s, 1 H). LCMS (Method 1): Rt 0.70 min., m/z 138.9 [M+H]⁺.

Step 4: 5-fluoro-1-methyl-6-oxo-1,6-dihydropyridine-2-carbonitrile (B)

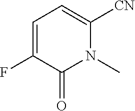

A mixture of 5-fluoro-6-oxo-1,6-dihydropyridine-2-carbonitrile (2.31 g, 16.73 mmol) and potassium carbonate (4.86 g, 35.2 mmol) in a 200 mL round bottom flask was treated with DMF (46 mL) and stirred for 15 minutes. MeI (1.2 mL, 19.19 mmol) was added and the mixture was stirred at room temperature for 45 minutes. The solvent was evaporated under reduced pressure. The residue was mixed with water (150 mL) and extracted with DCM (2×150 mL). The combined organic extracts were dried (MgSO₄), filtered, treated with silica gel, and evaporated under reduced pressure, then evaporated further at 60° C. under high vacuum. The material was chromatographed by Biotage MPLC with 0 to 35% EtOAc in hexanes, with isocratic elution at 16% EtOAc and 35% EtOAc while peaks were eluted. The peak that was eluted with 16% EtOAc was O-methylated material and was discarded. The peak that was eluted with 35% EtOAc provided the title compound B (1.70 g, 11.17 mmol, 67% yield) as a solid. ¹H NMR (300 MHz, DMSO-d₆): δ ppm 7.53 (dd, J=9.38, 7.62 Hz, 1 H), 7.18 (dd, J=7.77, 4.84 Hz, 1 H), 3.60 (s, 3 H). LCMS (Method 1): Rt 0.94 min., m/z 152.9 [M+H]⁺.

Step 5: (S)-5-((1-(6-chloro-2-oxo-1,2-dihydroquinolin-3-yl)ethyl)amino)-1-methyl-6-oxo-1,6-dihydropyridine-2-carbonitrile (Compound 1)

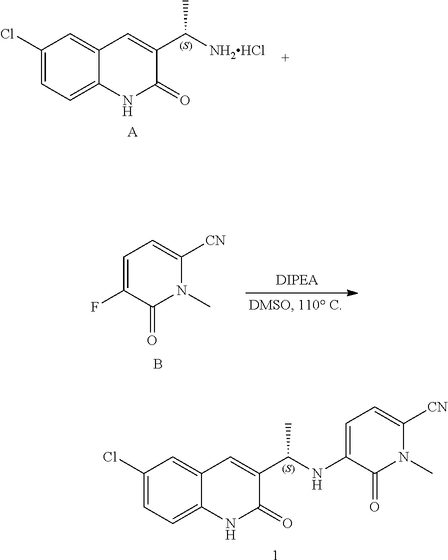

A mixture of 5-fluoro-1-methyl-6-oxo-1,6-dihydropyridine-2-carbonitrile B (1.23 g, 8.09 mmol), (S)-3-(1-aminoethyl)-6-chloroquinolin-2 (1H)-one hydrochloride A (1.91 g, 7.37 mmol) and N,N-diisopropylethylamine (3.8 mL, 21.8 mmol) in anhydrous dimethyl sulfoxide (57 mL) under N₂ was heated to 110° C. and stirred for 6 hours. After cooling to room temperature, the mixture was partitioned between EtOAc/H₂O (750 mL/750 mL). The organic layer was separated, dried (Na₂SO₄), and concentrated in vacuum. The residue was purified on an ISCO® chromatography system twice (40 g silica gel column, EtOAc/hexanes 0˜100%; 80 g silica gel column, MeOH/dichloromethane 0˜5%). The colorless fractions were combined and dichloromethane was removed under reduced pressure on rotavap until a lot of white solid precipitated out. The white solid was collected by filtration and washed with cold MeOH. It was then mixed with MeCN/H₂O (10 mL/25 mL) and lyophilized to afford the title compound 1 as a white solid (790 mg). m.p. 262-264° C. ¹H NMR (300 MHz, DMSO-d₆) δ: 12.07 (s, 1H), 7.75 (s, 1H), 7.73 (d, J=2.2 Hz, 1H), 7.51 (dd, J=8.6, 2.3 Hz, 1H), 7.31 (d, J=8.8 Hz, 1H), 6.97 (d, J=8.0 Hz, 1H), 6.93 (d, J=7.7 Hz, 1H), 5.95 (d, J=8.0 Hz, 1H), 4.68 (m, 1H), 3.58 (s, 3H), 1.50 (d, J=6.6 Hz, 3H). LCMS (Method 2): 100% pure@254 nm, Rt 10.78 min, m/z 355, 357 [M+H]+. The filtrate and the colored fractions (TLC pure) from the second ISCO were combined and treated with activated charcoal and filtered (until the filtrate is colorless). The filtrate was then concentrated under reduced pressure on rotavap to remove dichloromethane until a lot of white solid precipitated out. The white solid was collected by filtration and washed with cold MeOH. It was then mixed with MeCN/H₂O (10 mL/25 mL) and lyophilized to afford the title compound 1 as a white solid (970 mg). m.p. 262-264° C. ¹H NMR (300 MHz, DMSO-d₆) δ: 12.06 (s, 1H), 7.75 (s, 1H), 7.73 (d, J=2.5 Hz, 1H), 7.51 (dd, J=8.6, 2.3 Hz, 1H), 7.31 (d, J=8.8 Hz, 1H), 6.97 (d, J=8.0 Hz, 1H), 6.92 (d, J=8.0 Hz, 1H), 5.95 (d, J=8.0 Hz, 1H), 4.68 (m, 1H), 3.58 (s, 3H), 1.50 (d, J=6.9 Hz, 3H). LCMS (Method 2): 100% pure @ 254 nm, m/z 355, 357 [M+H]+.

### Example 2—Solid Form of (S)-5-((1-(6-chloro-2-oxo-1,2-dihydroquinolin-3-yl)ethyl)amino)-1-methyl-6-oxo-1,6-dihydropyridine-2-carbonitrile

Compound 1 can be prepared via the method described in Example 1. Compound 1 was dissolved in 18 volumes of dichloromethane (all volumes are with respect to the quantity of compound 1 (v/w)). The resulting solution was then concentrated under reduced pressure to approximately 5 volumes. To the mixture was added 5 volumes of ethyl acetate. The mixture was concentrated under reduced pressure to 5 volumes. To the mixture was added an additional 5 volumes of ethyl acetate, and the mixture again concentrated under reduced pressure to 5 volumes. The mixture was diluted to 10 volumes with ethyl acetate, and the mixture stirred at room temperature for 18 hours and then cooled to 0° C. The mixture was stirred at 0° C. for 3 hours and then filtered. The solids were rinsed with ethyl acetate and dried under vacuum (counterbalanced by nitrogen) at ambient temperature.

The crystalline solid was determined to be the solid form of Compound 1 Type A. The DVS Isotherm of Compound 1 Type A is shown in FIG. 1. DVS shows maximum water uptake of 0.25% w/w at 25° C./90% RH, indicating that Compound 1 Type A is not hygroscopic.

The thermal behavior Compound 1 Type A was evaluated using DSC. An endothermic event was observed at 256.6° C. (peak max). The onset temperature and heat of fusion were 255.0° C. and 108.7 J/g respectively (FIG. 2).

TGA data (FIG. 3) do not show significant release of moisture or nonaqueous residual volatiles from Compound 1 Type A.

The X-ray powder diffraction pattern of the crystalline Compound 1 Type A is depicted in FIG. 4, and the corresponding data is summarized in Table 2-1:

TABLE 2-1

| | d-spacing |
|---|---|
| 2 theta ± 0.2 | Å ± 0.2 |
| 5.7 | 15.4 |
| 6.3 | 14.0 |
| 8.5 | 10.4 |
| 10.6 | 8.4 |
| 11.4 | 7.8 |
| 12.8 | 6.9 |
| 13.8 | 6.4 |
| 14.2 | 6.2 |
| 15.2 | 5.8 |
| 15.6 | 5.7 |
| 17.3 | 5.1 |
| 17.9 | 5.0 |
| 18.2 | 4.9 |
| 18.9 | 4.7 |
| 19.6 | 4.5 |
| 20.6 | 4.3 |
| 21.5 | 4.1 |
| 22.0 | 4.0 |
| 22.8 | 3.9 |
| 23.6 | 3.8 |
| 24.5 | 3.6 |
| 24.8 | 3.6 |
| 25.3 | 3.5 |
| 25.6 | 3.5 |
| 26.0 | 3.4 |
| 26.3 | 3.4 |
| 27.0 | 3.3 |
| 27.8 | 3.2 |
| 28.9 | 3.1 |
| 30.0 | 3.0 |
| 31.2 | 3.0 |
| 32.1 | 2.8 |
| 33.6 | 2.7 |
| 34.1 | 2.6 |
| 36.3 | 2.5 |
| 37.0 | 2.4 |
| 38.1 | 2.4 |

### Example 3—Polymorph Screening

Polymorph screening for Compound 1 was conducted under different conditions, including anti-solvent addition, evaporation, slurry, solid/liquid vapor diffusion, crash cooling and grinding.

Slurry experiments were conducted at 4° C., RT and 50° C. in different solvent systems. For each experiment about 20 mg of Compound 1 Type A was suspended in 0.25-0.5 mL of solvent in a 1.5-mL glass vial. After the suspension was stirred for about one week at the desired temperature, the remaining solids were isolated for XRPD analysis. Results are summarized in Table 3-1.

TABLE 3-1

| Solvent, v/v | Temperature, ° C. | Solid Form |
|---|---|---|
| EtOH | RT | Type A |
| Acetone | RT | Type A |
| ACN | RT | Type A |
| 2-Me-THF/Heptane, 3:1 | RT | Type A |
| IPAc | RT | Type A |
| MTBE | RT/50 | Type A* |
| MIBK | RT/50 | Type A* |
| 1,4-Dioxane | RT | Type A |
| Toluene | RT | Type A |
| IPA | RT | Type A |
| Heptane | RT/50 | Type A* |
| H₂O | RT/50 | Type A* |
| EtOH/H₂O, 3:7 aw = 0.8 | RT | Type A |
| EtOH/H₂O, 9:1 aw = 0.5 | RT | Type A |
| EtOH/H₂O, 95:5 aw = 0.3 | RT | Type A |
| EtOH/H₂O, 985:15 aw = 0.1 | RT | Type A |
| Cyclopentylmethylether | 4 | Type A |
| DMF | 4 | Type A** |
| ACN/H₂O, 3:1 | 4 | Type A |
| DCM | 4 | Type A |
| MeOH | RT | Type A |
| EtOAc | RT | Type A |

*Type A observed at both RT (one week) and 50° C. (four days)
**Clear solution obtained at 4° C. stirring and solids obtained from evaporation at RT

Evaporation experiments were conducted under 12 conditions. For each experiment about 15 mg of Compound 1 Type A was dissolved in ˜1.0 mL of solvent in a 1.5-mL glass vial. The resulting clear solutions were subjected to slow evaporation at RT to induce precipitation. The solids, if observed, were isolated for XRPD analysis. The results are summarized in Table 3-2.

TABLE 3-2
Summary of evaporation experiments

| Solvent, v/v | Solid Form |
|---|---|
| ACN | Type A |
| MeOH | Type A |
| EtOAc | Amorphous |
| MEK | Type A |
| Acetone | Oil |
| DCM | Amorphous |
| THF | Amorphous |
| 1,4-Dioxane | Oil |
| Acetic acid | N/A |
| CHCl₃ | Amorphous |
| 2-Me-THF | Type A |
| MeOH/H₂O, 1:1 | Type A |

N/A: No solids obtained

A total of 8 anti-solvent addition experiments were carried out. About 5 mg of Compound 1 Type A was dissolved in 0.5-3.0 mL solvent to obtain a clear near saturated solution. 0.25-8.0 mL anti-solvent was then added to induce precipitation. The precipitate was isolated for XRPD analysis after stirring the resulting suspension overnight. Results are summarized in Table 3-3.

TABLE 3-3

| Solvent, v/v | Anti-solvent | Solid Form |
|---|---|---|
| MeOH | H₂O | Type A |
| DMSO | H₂O | Type A |
| ACN | Heptane | N/A |
| THF | Heptane | Type A |
| EtOAc | Heptane | Type A |
| MEK | Heptane | Type A |
| Acetone | Heptane | Type A |
| DCM | Heptane | Type A |

N/A: no solid obtained

Both solid vapor diffusion and solution vapor diffusion methods were used. Two solid vapor diffusion experiments were conducted. For each one, approximately 30 mg of Compound 1 Type A sample was weighted into a 3-mL vial, which was then placed into a 20-mL vial containing 4 mL of volatile solvent. The 20-mL vial was sealed and kept at RT for about two weeks allowing sufficient time for organic vapor of the solvents to interact with the solid sample. The solids thus obtained were isolated for XRPD test. Five solution vapor diffusion experiments were conducted. Approximate 15-30 mg of Compound 1Type A sample was dissolved in 2-4 mL of an appropriate solvent to obtain a saturated solution in a 5-mL vial. The solution was then placed into a 20-mL vial containing 4 mL of volatile solvents. The 20-mL vial was sealed and kept at RT for about two weeks allowing sufficient time for organic vapor of the anti-solvent to interact with the solution sample. The precipitates thus obtained were isolated for XRPD analysis. The results are summarized in Table 0-1 indicating no new form was observed.

TABLE 0-1
Summary of vapor diffusion experiments

| Method | Solvent, v/v | Anti-solvent | Solid Form |
|---|---|---|---|
| Solid vapor | H₂O | N/A | Type A |
| diffusion | DCM | N/A | Type A |
| Solution vapor | ACN | IPA | Amorphous |
| diffusion | MeOH | Toluene | Type A |
| | EtOAc | Heptane | Amorphous |
| | Acetone | Toluene | Type A |
| | THF | Heptane | Amorphous |

### Example 4—Formulations of Compound 1 Type A

Compound 1 Type A can be formulated into a form (e.g., a capsule or unit dosage form) for oral use.

Compound 1 Type A was formulated into capsules as summarized in Table 4-1. Each encapsulated drug product excipient meets the requirements of the respective current United States Pharmacopeia (USP) or National Formulary (NF) monograph. As permitted under EMA/CHMP/QWP/834816/2015, reference is made to the current compendial monographs in lieu of inclusion of the current compendial specifications. The capsule shells, which consist of gelatin and about 2.9% w/w of titanium dioxide (E171), are specified according to the current compendial requirements for each ingredient. Each excipient may be obtained from qualified suppliers that meet the cited specifications, and may be accepted upon a supplier certificate of analysis with minimal confirmatory identification testing upon receipt and periodic confirmation of supplier results.

TABLE 4-1

| Dose | | | Relative |
|---|---|---|---|
| Strength | Function | Component | weight² |
| 50 mg or | Active | Compound 1 Type A, Micronized¹ | 33.00 |
| 150 mg | Filler | Microcrystalline Cellulose NF/EP (Avicel PH101) | 61.12 |
| | Disintegrant | Croscarmellose Sodium NF/EP | 4.95 |
| | Lubricant | Magnesium Stearate NF/EP | 1.00 |
| | | Hard gelatin capsule shell, size 2 or size 00, white opaque | wt x |

¹20% excess Compound 1 Type A was micronized to obtain sufficient material needed for the batch.
²As used herein, relative weights (or % w/w) are given as a percentage relative to the total weight of the formulation.

Compound 1 Type A was formulated into tablets or capsules as summarized in Table 4-2.

TABLE 4-2

| | | Formulation | Formulation | Formulation | Formulation |
|---|---|---|---|---|---|
| | | 1 | 2 | 3 | 4 |
| Component | Function | (% w/w) | (% w/w) | (% w/w) | (% w/w) |
| Compound 1 | Active | 33.0 | 33.0 | 33.0 | 33.0 |
| Type A | | | | | |
| Avicel PH101 | Filler | 61.0 | 0 | 49.0 | 49.0 |
| (50 μm ) | | | | | |
| Avicel PH102 | Filler | 0.0 | 60.0 | 10.0 | 10.0 |
| (100 μm) | | | | | |
| Ac—Di-Sol | Disintegrant | 5.0 | 5.0 | 6.0 | 6.0 |
| Magnesium | Lubricant | 1.0 | 1.0 | 1.0 | 1.0 |
| Stearate | | | | | |
| Colloidal | Glidant/anti- | 0.0 | 1.0 | 1.0 | 1.0 |
| Silicon | adherent/ | | | | |
| Dioxide | anti-static | | | | |
| Manufacturing | — | Dry blending | Dry blending | Dry | Dry |
| Process | | | | granulation | granulation |
| Final Dosage | — | Capsule | Capsule | Capsule | Tablet |
| Form | | | | | |

1-20. (canceled)

21. A solid form of Compound 1:
Compound 1
characterized by an X-ray Powder Diffraction (XRPD) having diffractions at angles (2 theta+0.2) of 6.3, 23.6, and 27.8.

22. The solid form of claim 21 characterized by an X-ray Powder Diffraction (XRPD) having diffractions at angles (2 theta+0.2) of 6.3, 23.6, and 27.8, corresponding to d-spacing (angstroms±0.2) of 14.0, 3.8, and 3.2, respectively.

23. The solid form of claim 21 characterized by a differential scanning calorimetry (DSC) thermogram with an endothermic event observed at about 256.6° C.

24. The solid form of claim 22 characterized by a differential scanning calorimetry (DSC) thermogram with an endothermic event observed at about 256.6° C.

25. The solid form of claim 21 characterized by a dynamic vapor sorption (DVS) showing maximum water uptake of 0.25% w/w at 25° C./90% RH.

26. The solid form of claim 22 characterized by a dynamic vapor sorption (DVS) showing maximum water uptake of 0.25% w/w at 25° C./90% RH.

27. The solid form of claim 21, characterized by a DSC endothermic event at about 256.6° C., wherein the DSC is obtained by heating at 10° C./min from about 20° C. to about 300° C. using dry nitrogen to purge the system and the following parameters:

| Parameters | DSC |
|---|---|
| Pan Type | Aluminum pan, closed |
| Temperature | RT-250° C. |
| Ramp rate | 10° C./min |
| Purge gas | N₂ |

28. A solid form of olutasidenib, characterized by at least one of the following characteristics:
(i) an X-ray Powder Diffraction (XRPD) having diffractions at angles (2 theta±0.2) of 6.3, 23.6, and 27.8;
(ii) a differential scanning calorimetry (DSC) thermogram with an endothermic event observed at about 256.6° C.; and
(iii) a dynamic vapor sorption (DVS) showing maximum water uptake of 0.25% w/w at 25° C./90% RH.
